(19) United States
(12) Patent Application Publication
(10) Pub. No.: US 2009/0088729 A1
(43) Pub. Date: Apr. 2, 2009

(54) Vascular Access Devices Including A Tear-Resistant Septum

(75) Inventor: Marty L. Stout, South Weber, UT (US)

Correspondence Address: David W. Highet, VP & Chief IP Counsel;Becton, Dickinson and Company, (Kirton & McConkie), 1 Becton Drive, MC 110, Franklin Lakes, NJ, 07417-1880, US

(73) Assignee: BECTON, DICKINSON AND COMPANY, Franklin Lakes, NJ (US)

(21) Appl. No.: 11/863,089

(22) Filed: Sep. 27, 2007

Related U.S. Application Data
(60) Provisional application No. 60/828,357, filed on Oct. 5, 2006.

(51) Int. Cl. A61M 25/18

(52) U.S. Cl. 604/533

(57) ABSTRACT

A vascular access device may include a body, a septum at least partially disposed within the body, and/or a protective material adapted to resist tearing of the septum. A method of manufacturing a vascular access device may include disposing at least a part of a septum within a body and disposing a protective material in communication with the septum.

### RELATED APPLICATIONS

This application claims the benefit of U.S. Provisional Application No. 60/828,357, filed Oct. 5, 2006, entitled VASCULAR ACCESS DEVICE INCLUDING A TEAR-RESISTANT SEPTUM, which is incorporated herein by reference.

### BACKGROUND OF THE INVENTION

The present disclosure relates to infusion therapy with vascular access devices. Infusion therapy is one of the most common health care procedures. Hospitalized, home care, and other patients receive fluids, pharmaceuticals, and blood products via a vascular access device inserted into the vascular system. Infusion therapy may be used to treat an infection, provide anesthesia or analgesia, provide nutritional support, treat cancerous growths, maintain blood pressure and heart rhythm, or many other clinically significant uses.

Infusion therapy is facilitated by a vascular access device. The vascular access device may access a patient's peripheral or central vasculature. The vascular access device may be indwelling for short term (days), moderate term (weeks), or long term (months to years). The vascular access device may be used for continuous infusion therapy or for intermittent therapy.

A common vascular access device is a plastic catheter that is inserted into a patient's vein. The catheter length may vary from a few centimeters for peripheral access to many centimeters for central access. The catheter may be inserted transcutaneously or may be surgically implanted beneath the patient's skin. The catheter, or any other vascular access device attached thereto, may have a single lumen or multiple lumens for infusion of many fluids simultaneously.

Vascular access devices commonly include a Luer adapter, or other connector or adapter, to which other medical devices may be attached. For example, an IV (intravenous) administration set may be attached to a vascular access device to provide a fluid conduit for the continuous infusion of fluids and pharmaceuticals from an intravenous (IV) bag. A variety of medical devices may cooperate with vascular access devices to provide selective, temporary, or long-term access to the vascular system of a patient. A vascular access device may include a body having a lumen therethrough and a septum for selectively closing the lumen. The septum may be opened with a blunt cannula, a male Luer of a medical device, or other suitable medical device.

Vascular access devices provide many significant benefits to patients and medical practitioners. A vascular access device is most beneficial to patients when the septum forms a proper seal between the accessed vascular system and the outside or external environment. In an ideal vascular access device, the septum would continuously seal the patient's vascular system, which may include external vascular equipment intentionally coupled to the patient's internal vascular system by a medical practitioner, from the external environment.

As with most systems, one of the biggest challenges to the proper function of the vascular access device is when there is a change in the system, such as when different medical devices are connected or disconnected from the vascular access device. If the seal against the external environment is broken during the connection or disconnection of a medical device, there is the possibility of infection being introduced into the patient's vascular system. Additionally, if a pressure difference is created across the vascular access device, there becomes the possibility that blood will be drawn up the catheter system and possibly into the vascular access device or beyond. Alternatively, a pressure difference across the vascular access device may make it more difficult to couple other medical devices to the vascular access device.

As introduced above, vascular access devices are often coupled with a blunted cannula, such as the tip of a syringe, with a male Luer connector, or with other medical devices. These medical devices may be coupled to the vascular access devices by pressing a portion of the medical device into a slit or passage in the septum. Some medical devices are coupled to the vascular access device through a twisting motion by which the body or other portion of the medical device is coupled to the body of the vascular access device and by which a portion of the medical device is disposed in the slit or passage of the septum. Other methods of coupling the vascular access device to one or more medical devices may be used as well.

Regardless of the methods used to couple medical devices to the vascular access device, repeated transitions of the septum between open and closed configurations applies stress to the septum. In some experiences the septum has been seen to tear, either slightly or more significantly, at the edges of the slit that allows other devices to access the internal vascular system through the lumen of the body. In previous vascular access devices, two common tear patterns have been observed: radial tearing and circumferential tearing. Depending on the nature of the tear, the impacts of the tear may include a decrease in the quality of the seal formed by the septum or pieces or particles of the septum breaking free from the remainder of the septum. In any event, a septum that is modified from the manufacturer's intended and safety-tested design is not preferred for a number of reasons. The present disclosure is directed to vascular access devices, and methods of manufacturing vascular access devices, that include a tear-resistant septum.

### BRIEF SUMMARY OF THE INVENTION

A vascular access device may include a body, a septum at least partially disposed in the body, and/or a protective material adapted to resist tearing of the septum. The body may define a lumen extending through the body. The septum may at least substantially seal the lumen extending through the body and may include a top surface. The top surface may communicate with the protective material.

The protective material may be formed as at least one raised surface above the top surface of the septum. The body may also include an upper end and the septum may include a bottom surface in communication with the upper end. The protective material may be located on the top surface of the septum opposite the bottom surface that is in communication with the upper end of the body.

The protective material may form multiple protective structures exposed on the top surface of the septum. The protective material may be formed of the same material as the septum or of a material different from the material forming the septum. The multiple protective structures may distribute load throughout the septum when a load is placed upon the multiple protective structures.

The protective material may be adapted to be secured to the body of the vascular access device. The protective material may include a protective cap. The body may include threads, and the protective cap may secure to the threads of the body. The protective cap may be formed of a rigid material.

The protective cap may define a lumen extending through the protective cap, and the lumen extending through the protective cap may align with a lumen extending through the body when the protective cap is secured to the body. The surface of the protective cap that defines the lumen extending through the protective cap may be tapered. In one embodiment the surface of the protective cap may be tapered to narrow at approximately a six degree angle as the tapered lumen approaches the septum.

A method of manufacturing a vascular access device may include providing a body having a first body end region and a second body end region. The body may define a passage extending through the body. The method may also include providing a septum having a top surface, disposing at least a part of the septum within the first body end region, providing a protective material, and/or disposing the protective material in communication with the top surface of the septum.

The method may also include resisting tearing of the septum with the protective material. Disposing the protective material in communication with the top surface of the septum may include forming the protective material from the material of the septum, over-molding the septum with the protective material, and/or mechanically securing the protective material to the body.

A vascular access device may include a body means, a sealing means, and/or a means for discouraging tearing of the sealing means. The body means may be provided for selective coupling to a vascular system of a patient and to at least one additional medical device. The body means may have a passage extending therethrough. The sealing means may include a slit for selectively and at least substantially sealing the passage through the body. The means for discouraging tearing of the sealing means may be in communication with the sealing means.

These and other features and advantages of the present disclosure may be incorporated into vascular access devices and will become more fully apparent from the following description and appended claims, or may be learned by the practice and implementation of the present disclosure. As described above, the present disclosure does not require that all of the features described herein be incorporated into every embodiment nor is it required that certain features be used exclusive of other features. Vascular access devices within the scope of the present disclosure may include one or more combinations of the features described herein.

### BRIEF DESCRIPTION OF THE SEVERAL VIEWS OF THE DRAWINGS

In order that the above-recited and other features and advantages of the disclosure may be readily understood, a more particular description is provide below with reference to the appended drawings. These drawings depict only exemplary embodiments of vascular access devices according to the present disclosure and are not therefore to be considered to limit the scope of the disclosure.

FIG. 1 is a perspective view of an extravascular system connected to the vascular system of a patient.

FIG. 2 is a top view of a vascular access device.

FIG. 3 is a perspective side view of a vascular access device.

FIG. 4 is a cross section view of a vascular access device.

FIG. 5 is a cross section view of a vascular access device with the cross section being 90 degrees offset from the cross section of FIG. 4.

FIG. 6 is a top view of a septum and a protective material.

FIG. 7 is a cross section view of the septum of FIG. 6 taken along lines A-A of FIG. 6.

FIG. 8 is a cross section view of the septum of FIG. 7 having a separate device inserted therein.

FIG. 9 is a cross section view of a vascular access device secured to a protective material.

### DETAILED DESCRIPTION OF THE INVENTION

It will be readily understood that the components of the present disclosure, as generally described and illustrated in the figures herein, could be arranged and designed in a wide variety of different configurations. Thus, the following more detailed description, as represented in the figures, is not intended to limit the scope of the disclosure, but is merely a representative of exemplary combinations of the components.

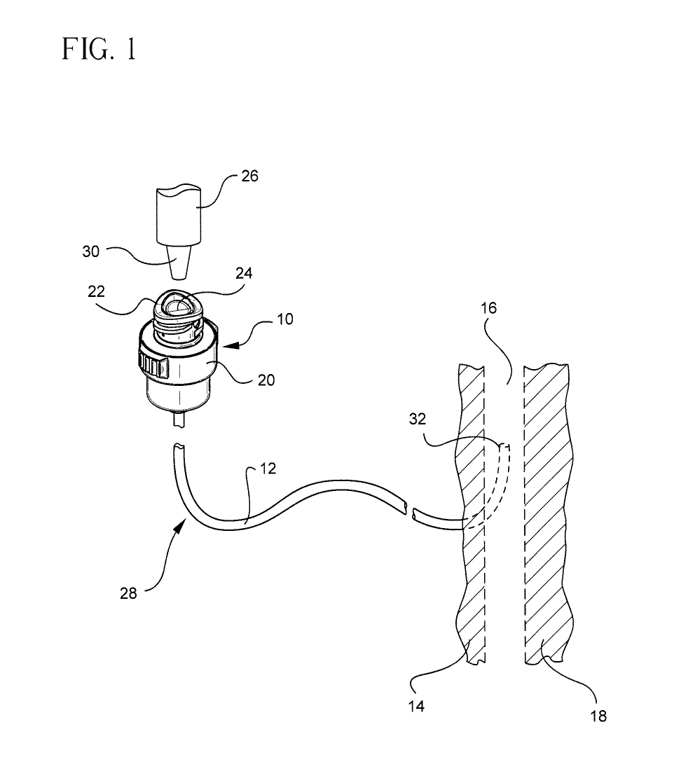

Referring now to FIG. 1, a vascular access device 10 is used to introduce a substance via a catheter 12 across the skin 14 and into a blood vessel 16 of a patient 18. The vascular access device 10 includes a body 20 with a lumen and a septum 22 placed within the lumen. The vascular access device 10, including the body 20 and the septum 22, will be more thoroughly described with reference to the remaining figures where particular features are better illustrated. As shown in FIG. 1, the septum 22 has a slit 24 through which a separate extravascular device 26, such as a syringe, may introduce a substance into the vascular access device 10. A syringe is one exemplary separate device 26. Other suitable extravascular devices may include additional vascular access devices, IV administration sets, or other common or yet to be developed medical devices.

The device 10 and all structures used in combination therewith may form a larger extravascular system 28. As part of operating the extravascular system 28, a tip 30 of the separate device 26 may be inserted into the vascular access device 10 through the slit 24 of the septum 22. The tip 30 penetrates the device 10 separating at least portions of the two opposing slit surfaces of the septum 22. The septum 22 and the slit 24 may be configured to seal, or at least substantially seal, around the tip 30 as it is inserted into the vascular access device 10. Accordingly, the surfaces near the slit ends may not be separated until the tip 30 is sufficiently inserted into vascular access device 10. The tip 30 serves to open the slit 24 to allow fluid to pass through the device 10, into the catheter 12, and out the end 32 of the catheter when the device is in use.

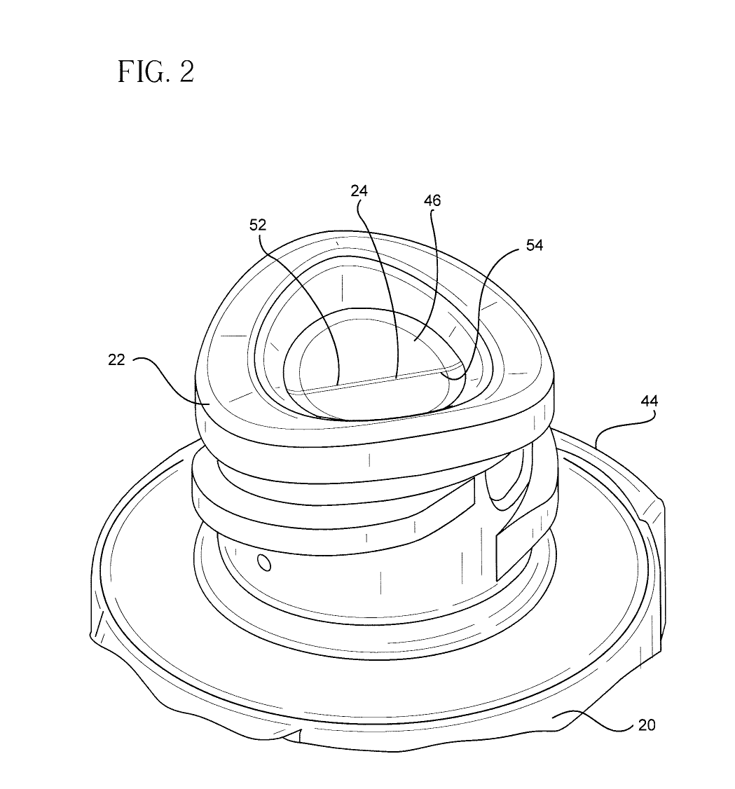

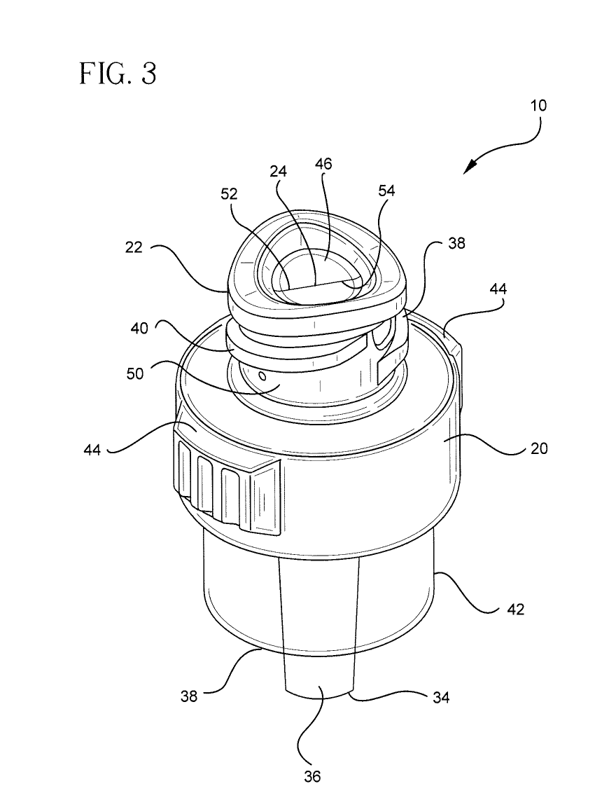

The features of an example of a vascular access device 10 are illustrated in FIGS. 2 and 3. As illustrated in these figures, the septum 22 includes a portion that extends beyond the body 20 but is otherwise disposed substantially within the body 20. The body 20 may include a cannula 34 for coupling with a catheter or other medical device. The cannula 34, along with other components of the body 20, may cooperate to form a lumen 36 through the body 20. The body 20 may also include connection regions 38, such as female Luer connector 40 or male Luer connector 42, to enable the vascular access device to be selectively coupled to other medical devices. Additionally, the body 20 may include grips 44, which may be ridges or other structures on the surface of the body 20, to facilitate the manipulation of the vascular access device 10. The body 20 may include other features or structures common to vascular access devices.

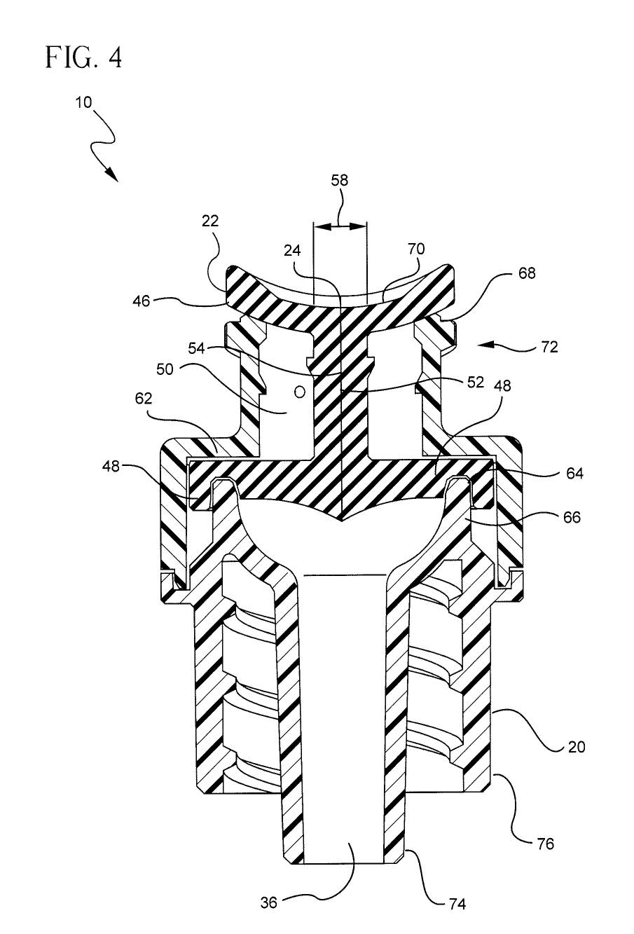

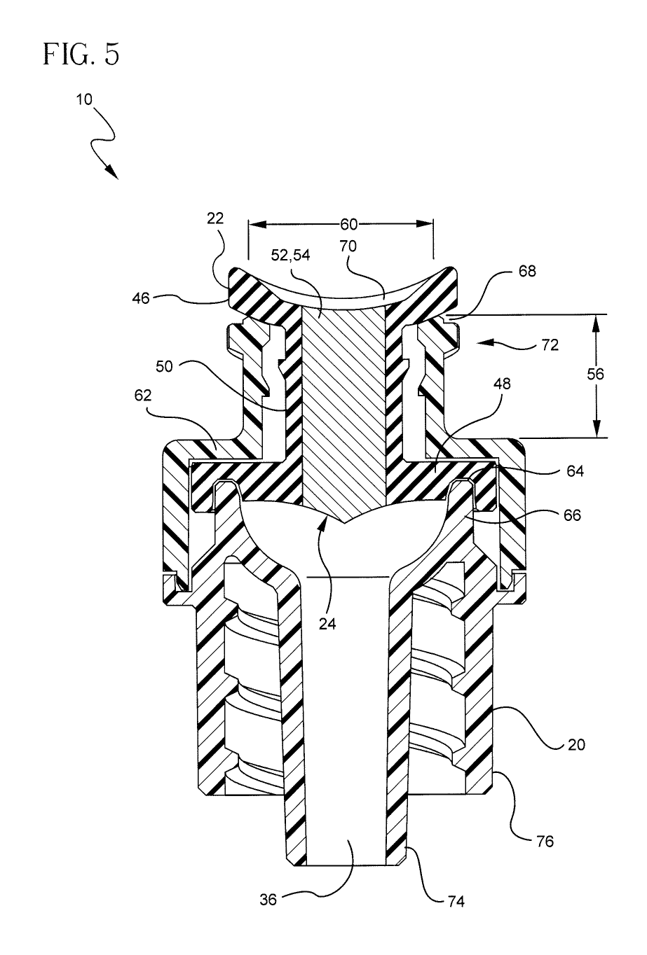

With continuing reference to FIGS. 2 and 3, the septum 22 is substantially disposed within the body 20 of the vascular access device 10. More specifically, the septum 22 includes a top disk 46, a bottom disk 48, and a throat region 50 extending between the top disk 46 and the bottom disk 48. The throat section 50 and bottom disk 46 are more clearly visible in the cross section view presented in FIGS. 4 and 5. As used herein, the top disk 46 may also be referred to as a saddle 46 and the bottom disk 48 may be referred to as an anchor 48. With more particular reference to FIG. 2, the septum 22 is shown to include a slit 24 having opposing slit surfaces 52, 54. As described above, the opposing slit surfaces 52, 54 of the slit 24 are moved apart to open the slit when the tip 30 of a medical device is inserted into a vascular access device 10.

Referring now to FIGS. 4 and 5, cross sectional views of a vascular access device 10 are shown to better illustrate particular aspects of an exemplary septum 22. As illustrated, FIGS. 4 and 5 are cross sections of the same vascular access device with the cross sections being taken along orthogonal lines of cross section. FIG. 4 illustrates a vascular access device 10 showing the throat region 50 spanning between the saddle 46 and the anchor disk 48. The throat region 50 may have any suitable length 56 between the saddle 46 and the anchor 48, which length 56 may vary to accommodate the configuration of the body 20. As one example, the length 56 may be selected to position the anchor disk 48 within the body 20 and the saddle 46 outside the body, as illustrated.

The throat region 50 also has a thickness 58, shown in FIG. 4, and a width 60, shown in FIG. 5. The width 60 and thickness 58 of the throat region 50 may be selected to meet the needs of the medical practitioner and the vascular access device 10 in which the septum 22 is being incorporated. The width 60 may be selected to provide sufficient room for a slit 24 sufficiently wide to accommodate the desired tips 30 of the cooperating medical devices 26. The thickness 58 of the throat region 50 may be selected to provide sufficient strength to the throat region while still providing sufficient elasticity and/or flexibility to allow the slit surfaces 52, 54 to separate as the tips 30 are inserted into the vascular access device 10.

The bottom disk 48, or anchor disk, may be configured to have a size, such as a diameter, that is selected to fit within the body 20 and to be retained in the body by a shoulder region 62. Additionally or alternatively, the bottom disk 48 may be anchored within the body 20 through other means, such as through adhesives or fasteners. As illustrated in FIGS. 4 and 5, the bottom disk 48 may include one or more grooves or slots 64 that may be adapted to cooperate with portions of the body 20 to further anchor the septum 22 in place. The bottom disk 48 and one or more portions of the body 20 may be configured to anchor the septum 22 rotationally within the body, longitudinally within the body, and/or laterally within the body. As one example, fingers 66 of the body 20 may be adapted to fit in the grooves 64 to prevent lateral movement and/or rotational movement of the septum 22. Additionally or alternatively, the fingers 66 may be sized to press the bottom disk 48 into the shoulder region 62 so that the top surface of the bottom disk is in contact with the body 20. As one example, the fingers 66 may cause the bottom disk 48 and the body 20 to form a seal. In addition to the features described, the bottom disk 48 may include additional features or elements customary for vascular access devices.

FIGS. 4 and 5 illustrate that the top disk 46 may be configured to be disposed outside of the body 20. As illustrated the bottom surface of the top disk 46 rests on the upper end 68 of the body 20. FIG. 4 further illustrates that the top disk 46 may be configured to provide a well 70 or indentation. The well 70 may assist in guiding the tip 30 of the cooperating medical device 26 into the slit 24 of the vascular access device 10. As seen in FIGS. 4 and 5, the well 70, in some implementations, may cause the top disk 46 resemble a saddle. The well 70, when present, may be formed by thinning a portion of the top disk 46 and/or by applying upward pressure to the outside edge of the top disk 46. As one example, the septum 22 may be configured with a throat region 50 that is minimally shorter than the distance between the shoulder region 62 of the body 20 and the upper end 68 of the body. Accordingly, the septum material of the throat region 50 and the top disk 46 may be slightly stretched by this difference causing the top disk to flex forming the well 70. The well 70 may be formed in other suitable manners.

As discussed above and as illustrated in FIG. 4, the top disk 46 contacts the upper end 68 of the body 20. The interface between the top disk 46 and the upper end 68 of the body 20 may form an additional seal, which may be similar to the seal between the bottom disk 48 and the body 20. Additionally or alternatively, an adhesive may be used to bond the top disk 46 to the upper end 68 of the body. Moreover, structural features, such as grooves, may be incorporated into the bottom surface of the top disk 46 to cooperate with the body 20 to form a seal. The seals formed by the top disk 46 and/or the bottom disk 48 and the body 20 are adapted to seal, or at least substantially seal the lumen 36 through the body 20. Moreover, when the slit surfaces 52, 54 are together (i.e., not separated by a tip 30 and not otherwise separated by tears, cracks, or other modifications to the septum), the septum 22 seals, or at least substantially seals the passage through the lumen of the body 20.

For purposes of description, the upper end 68 of the body 20 and the portions adjacent thereto may be referred to as a first body end region 72 whereas the lower end 74 of the body 20 and the portions adjacent thereto may be referred to as the second body end region 76. The use of the terms first and second to denominate the end regions, or other elements described herein, is not meant to imply any order between the two end regions but merely to distinguish between the two. While the terms top and bottom are also used herein to designate and distinguish features, components, or parts of the vascular access device, it should be understood that the orientation of the vascular access device may change during use of the device; accordingly, the terms top and bottom are not intended to be limiting with respect to orientation during use of the device but are referencing relative locations in the figure being discussed.

The body 20 and the septum 22 may be constructed of a variety of suitable materials. Commonly, the body 20 of the vascular access device 10 will be made of a plastic, and preferably a plastic material that facilitates molding the body. As illustrated in FIGS. 4 and 5, the body 20 is formed from two pieces that are molded or adhered together to form the body once the septum 22 is in place. Other methods and materials may be used for manufacturing the body 20, some of which may be currently practiced and some of which may be developed in the future.

Similarly, the septum 22 may be made of a variety of suitable materials and through a variety of suitable manufacturing methods. For example, the septum may be formed from liquid silicone rubber through suitable molding procedures, such as insert molding, injection molding, other molding techniques, or a combination of molding techniques. The materials and manufacturing methods used may vary in order to achieve the objectives of the invention as claimed. The objectives of the present invention include, but are not limited to or required to include the following: resisting tearing of the septum 22 such as tearing that may occur in the top disk 46, saddle 70, or any region or material adjacent thereto.

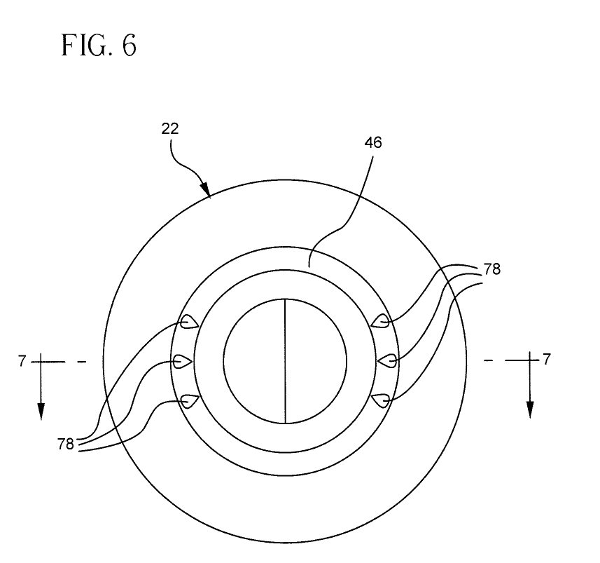

Referring now to FIG. 6, a top view of the top disk 46 of the septum 22 is shown. The top surface of the top disk 46 includes a protective material 78 that is adapted to resist tearing of the septum 22. The protective material 78 may form multiple protective structures exposed on the top surface of the top disk 46 of the septum 22. The multiple protective structures may be employed in order to distribute the load that is placed upon the top surface of the top disk 46 of the septum 22 when a separate device 26 accesses the vascular access device 10.

The protective material 78 may be formed of the same material as the septum 22, or may be formed of a material that is different from the material forming the septum 22. The materials forming the protective material 78, for example, may include the same liquid silicone rubber that may be used to form the septum 22 and/or a different material, such as a rigid plastic that is over-molded on and/or in the septum 22.

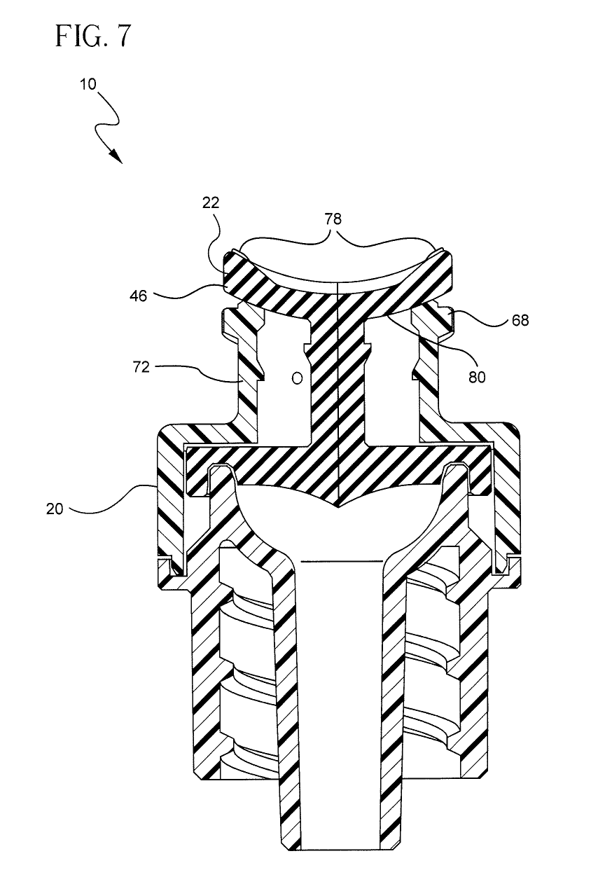

Referring now to FIG. 7, a cross section view of FIG. 6 taken along lines A-A is shown. The cross section view reveals the protective material 78 on the top surface of the top disk 46 of the septum 22. The septum 22 is secured to the upper end 68 of the body 20, which forms a part of the first body end region 72. The protective material 78 is located on the top surface of the top disk 46 of the septum 22 in a position that is opposite the bottom surface 80 of the top disk 46, which bottom surface 80 is in communication with the upper end 68 of the body 20.

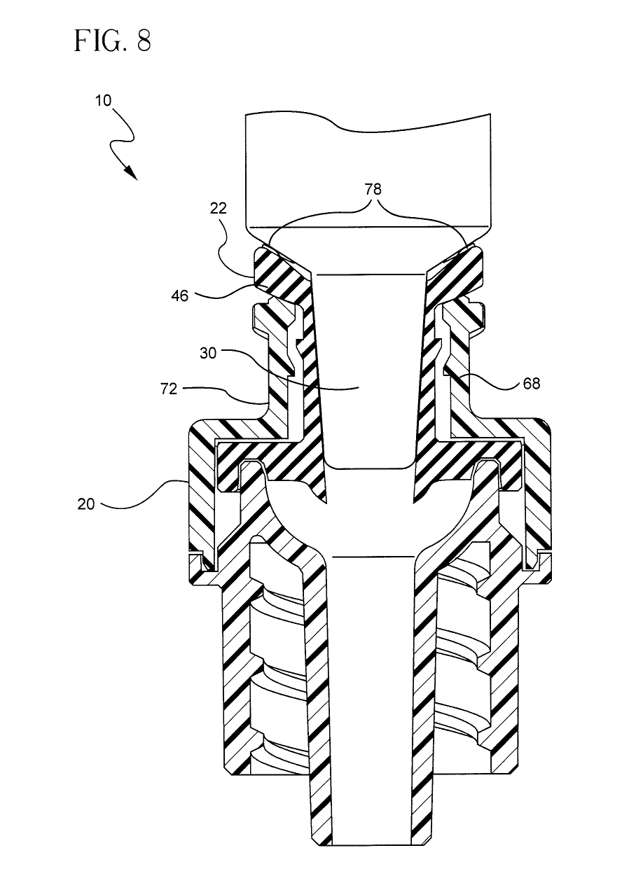

Referring now to FIG. 8, a cross section view of the vascular access device 10 including a septum 22 having a protective material 78 thereon as described with reference to FIGS. 6 and 7 is further illustrated in FIG. 8 being accessed by the tip 30 of a separate device 26. Upon full insertion of the tip 30 of a separate device 26 into the septum 22 of the device 10, the progression of the device 26 into the septum 22 will be halted when the structure of the separate device 26 comes into contact with the protective material 78 that is located on the top surface of the septum 22.

The protective material 78 serves as protection, a buffer, a bumper, or another insulative member capable of protecting the remaining body of the top disk 46 of the septum 22 when the material of the top disk 46 is compressed towards the rigid material of the upper end 68 of the body 20. Absent the material 78, the rigid material of the separate device 26 would compress the soft material of the top disk 46 against the rigid material of the body 20, causing the soft material of the top disk 46 to rip, tear, be pinched, or otherwise be damaged between the rigid materials of the separate device 26 and body 20. The protective material 78 serves to provide additional structure to the softer material of the top disk 46 in an area where the softer material is most vulnerable and likely to be damaged as a result of the force and load placed upon the device 10 by the separate device 26.

Multiple bumps or structures of the protective material 78 may be placed at any number of points or locations in communication with the material of the top disk 46. For example, three bumps of protective material 78 have been placed on opposite sides of the top disk 46 as best illustrated in FIG. 6. The multiple bumps of the protective material 78 serve to distribute the load among the bumps and throughout the material of the top disk 46 such that the force exerted upon any given point of the top disk 46 by the separate device 26 is less likely to damage a specific location in the material of the top disk 46. Since various types, materials, and shapes of separate vascular access devices 26 may be inserted into the device 10, any number of corresponding arrangements, materials, and locations of protective materials 78 may be employed to adequately distribute the load and protect the material of the top disk 46 from tearing.

In addition to providing protection to the material of the septum 22, the protective material 78 also provides tactile feedback to an operator inserting the separate device 26 into the device 10. The tactile feedback enables the operator to know when the tip 30 of the separate device 26 is fully inserted into device 10. Upon learning that the device 26 is fully inserted, the operator may then choose to cease forcing the tip 30 against the top disk 46 in order to prevent any potential damage due to unnecessary force.

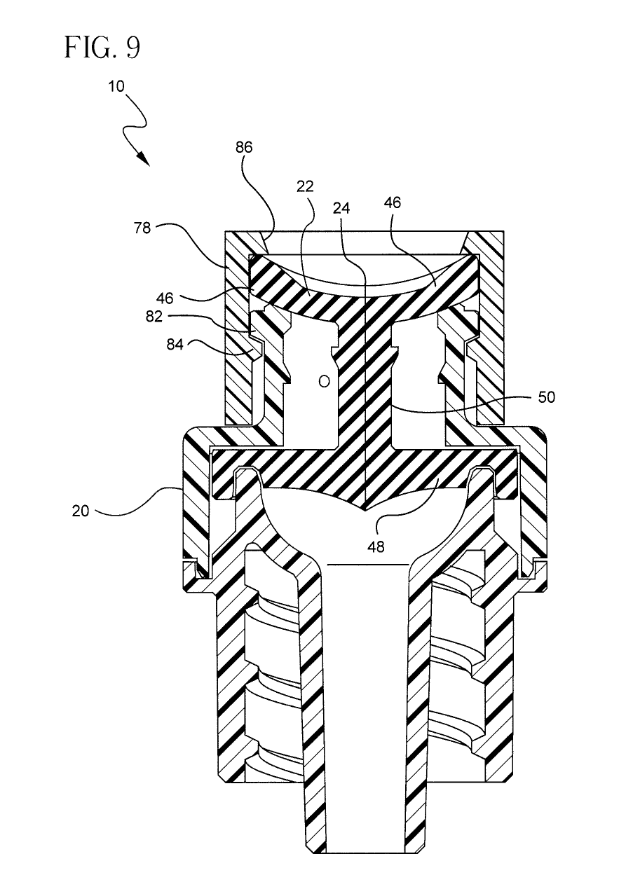

Referring now to FIG. 9, a vascular access device 10 is shown secured to a protective material 78. The protective material 78 is adapted to be secured to the body 20 of the vascular access device 10 by engaging the threads 82 of the body 20 with corresponding threads 84 on the protective material 78. Any means of mechanical or other attachment in addition or alternate to threads may be used to secure the body 20 and the protective material 78 together. The protective material 78 is formed in the shape of a protective cap that may be screwed onto the device 10 where an eccentric syringe tip or Luer slip is likely to be used as the separate device 26 for access into the device 10. Such protective cap will protect the material of the top disk 46 from damage as the device 10 is accessed by the separate device 26, including the insertion of any tip along an axis which is not parallel to the axis of the slit 24.

The protective cap of the protective material 78 is formed of a rigid material and defines a lumen extending through the protective cap. The lumen of the protective cap aligns with the lumen extending through the body 20 of the device 10 when the protective cap is secured to the body 20. The protective cap of the protective material 78 includes a surface 86 defining the lumen. The surface 86 is tapered. In one embodiment the surface is tapered to narrow at a six degree angle as the tapered lumen approaches the septum 22. This taper will direct the tip of a separate device 26 towards the slit 24 and will ensure that the edges of the top disk 46 are protected from tearing or damage. The size and angle of the taper and the hole formed by the taper also act as an interference fit with the tip 30 of a separate device 26 to ensure that the tip 30 is not over-inserted to the point of pinching, tearing, or otherwise damaging the septum 22 or the body 20.

The protective cap of the protective material 78 also provides additional advantages when secured to a device 10. First, the protective cap, when formed of a rigid material, may serve as a stop to prevent over-insertion of the tip 30 of a separate device 26 into the device 10. The tapered surfaces 86 also serve to ensure that the tip 30 of a separate device 26 is inserted directly through the center of the slit 24, preventing any damage to the column or throat section 50 that would occur if a tip 30 were to force the material of the throat section 50 against any portion of the body 20. The stopping feature of the protective cap will also prevent tears to the bottom disk 48 which may result from over-insertion of the tip 30 of the device 26 into the device 10.

The embodiments described with reference to FIGS. 6 through 9 thus provide a means for discouraging tearing of the septum or sealing means in the form of a protective material.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Where the disclosure, the presently filed claims, or subsequently filed claims recite “a” or “a first” element or the equivalent thereof, it should be within the scope of the present inventions that such disclosure or claims may be understood to include incorporation of one or more such elements, neither requiring nor excluding two or more such elements.

Applicants submit claims herewith and reserve the right to submit claims directed to certain combinations and subcombinations that are directed to one of the disclosed inventions and are believed to be novel and non-obvious. Inventions embodied in other combinations and subcombinations of features, functions, elements and/or properties may be claimed through amendment of those claims or presentation of new claims in that or a related application. Such amended or new claims, whether they are directed to a different invention or directed to the same invention, whether different, broader, narrower or equal in scope to the original claims, are also regarded as included within the subject matter of the inventions of the present disclosure.

1. A vascular access device, comprising:
a body defining a lumen extending therethrough; and
a septum at least partially disposed in the body to at least substantially seal the lumen extending through the body, wherein the septum includes a top surface, wherein the top surface communicates with a protective material, and wherein the protective material is adapted to resist tearing of the septum.

2. The vascular access device of claim 1, wherein the protective material is formed as at least one raised surface above the top surface of the septum.

3. The vascular access device of claim 1, wherein the body includes an upper end, wherein the septum includes a bottom surface in communication with the upper end, and wherein the protective material is located on the top surface of the septum opposite the bottom surface that is in communication with the upper end of the body.

4. The vascular access device of claim 3, wherein the protective material forms multiple protective structures exposed on the top surface of the septum.

5. The vascular access device of claim 4, wherein the protective material is formed of the same material as the septum.

6. The vascular access device of claim 4, wherein the protective material is formed of a material different from the material forming the septum.

7. The vascular access device of claim 4, wherein the multiple protective structures distribute load throughout the septum when a load is placed upon the multiple protective structures.

8. The vascular access device of claim 1, wherein the protective material is adapted to be secured to the body.

9. The vascular access device of claim 8, wherein the protective material includes a protective cap.

10. The vascular access device of claim 9, wherein the body includes threads, and wherein the protective cap secures to the threads of the body.

11. The vascular access device of claim 10, wherein the protective cap is formed of a rigid material.

12. The vascular access device of claim 11, wherein the protective cap defines a lumen extending therethrough, and wherein the lumen extending through the protective cap aligns with the lumen extending through the body when the protective cap is secured to the body.

13. The vascular access device of claim 12, wherein the surface of the protective cap defining the lumen extending therethrough is tapered.

14. The vascular access device of claim 13, wherein the surface of the protective cap defining the lumen extending therethrough is tapered to narrow at a six degree angle as the tapered lumen approaches the septum.

15. A method of manufacturing a vascular access device, comprising:
providing a body having a first body end region and a second body end region and defining a passage extending therethrough;
providing a septum having a top surface;
disposing at least a part of the septum within the first body end region;
providing a protective material; and
disposing the protective material in communication with the top surface of the septum.

16. The method of claim 15, further comprising resisting tearing of the septum with the protective material.

17. The method of claim 15, wherein disposing the protective material in communication with the top surface of the septum includes forming the protective material from the material of the septum.

18. The method of claim 15, wherein disposing the protective material in communication with the top surface of the septum includes overmolding the septum with the protective material.

19. The method of claim 15, wherein disposing the protective material in communication with the top surface of the septum includes mechanically securing the protective material to the body.

20. A vascular access device, comprising:
a body means for selective coupling to a vascular system of a patient and to at least one additional medical device and having a passage extending therethrough;
a sealing means including a slit for selectively and at least substantially sealing the passage through the body; and
means for discouraging tearing of the sealing means in communication with the sealing means.
